(19) United States
(12) Patent Application Publication
(10) Pub. No.: US 2025/0352491 A1
(43) Pub. Date: Nov. 20, 2025

(54) NANOPARTICLES FOR ENHANCED TRANSPORT ACROSS LYMPHATIC BARRIERS

(71) Applicant: University of Maryland, College Park, College Park, MD (US)

(72) Inventors: Katharina Maisel, Rockville, MD (US); Jacob McCright, Gaithersburg, MD (US)

(73) Assignee: University of Maryland, College Park, College Park, MD (US)

(21) Appl. No.: 19/208,028

(22) Filed: May 14, 2025

Related U.S. Application Data
(60) Provisional application No. 63/647,805, filed on May 15, 2024.

(51) Int. Cl. A61K 9/51; A61K 39/385

(52) U.S. Cl. CPC A61K 9/5169; A61K 9/5146; A61K 39/385

(57) ABSTRACT

The present disclosure is drawn to nanoparticles, method of using and making thereof. The disclosed nanoparticles comprise nanoparticles, that may be rod-shaped and/or coated on the outer surface with albumin, that are designed to effectively cross lymphatic barriers as an effective strategy to improve the efficacy and translatability of putative immunotherapies. Delivery systems or pharmaceutical compositions comprising the disclosed nanoparticles are also provided.

This application claims priority to U.S. Provisional Application Ser. No. 63/647,805 filed on May 15, 2024, the entire contents of which are incorporated herein in its entirety.

### GOVERNMENT SUPPORT STATEMENT

This invention was made with government support under GM142835 awarded by the National Institutes of Health, and DMR2047017 awarded by the National Science Foundation. The government has certain rights in the invention.

### TECHNICAL FIELD

The present disclosure is drawn to nanoparticles, method of using and making thereof. The disclosed nanoparticles comprise nanoparticles, that may be rod-shaped and/or coated on the outer surface with albumin, that are designed to effectively cross lymphatic barriers as an effective strategy to improve the efficacy and translatability of putative immunotherapies. Delivery systems or pharmaceutical compositions comprising the disclosed nanoparticles are also provided.

### BACKGROUND

Therapeutic treatments targeting the immune system are desirable. These treatments include classic vaccines as well as cancer immunotherapies. Recently, lymphatic vessels have received significant attention as a potential drug delivery target for immunotherapies. Lymphatics form the body's natural conduit between peripheral tissues and the local draining lymph nodes, where the adaptive immune response is shaped. Delivery of such immunotherapies to the draining lymph nodes, where adaptive immunity is formed, amplifies their efficacy, thus potentially improving their clinical outcomes. Growing interest in reaching lymph nodes with immunotherapies has sparked interest in targeting lymphatics with nanoparticles, but the mechanisms that lymphatics use to transport nanoparticles has remained largely unknown. Additionally, there exists little research on the nanoparticle properties required to cross the lymphatics. Accordingly, there is a need for rational material design approaches to engineering nanoparticles for improved lymphatic delivery.

### SUMMARY

The present disclosure provides novel nanoparticles, and methods of using and making thereof. More specifically, the provided nanoparticles comprise (i) an outer surface comprising albumin; and/or a rod-shaped nanoparticle. In a specific embodiment, the nanoparticle may further comprise a coating of polyethylene glycol (PEG). Nanoparticles that are rod-shaped and/or coated on the outer surface with albumin, have been shown herein to effectively cross lymphatic barriers and can therefor be used as an effective strategy to improve the efficacy and translatability of putative immunotherapies.

In certain embodiments, the inner core of the inventive nanoparticle comprises a biocompatible and/or a synthetic material. In a specific embodiment, the nanoparticle is formed from a biocompatible polymer. In a specific embodiment the nanoparticle is a nanoliposome. In certain embodiments, the diameter of the nanoparticle is about 50 nm to about 500 nm.

The present disclosure further provides that the inventive nanoparticle, having an albumin coating and/or a rod-shape, comprises a payload that can be located in any place inside or on the surface of the nanoparticle. As used herein, rod-shape refers to a nanoparticle having an aspect ratio of >1. In certain embodiments, the payload comprises one or more therapeutic agents, prophylactic agents, diagnostic agents, or a combination thereof. In certain embodiments, the payload is a metallic particle, a polymeric particle, a dendrimer particle, or an inorganic particle. In a specific embodiment, the payload comprises an antigen or hapten for stimulation of a protective immune response. In another embodiment, the payload comprises an allergen or self-antibody for use in development of tolerance to said allergen or self-antibody.

In some embodiments, the payload is coated on the nanoparticle using a crosslinking agent. In some embodiments, the payload is adsorbed onto the nanoparticle surface. In some embodiments, the payload is adsorbed onto the nanoparticle surface followed by covalent crosslinking of the payload to the nanoparticle surface using a crosslinking agent.

The provided nanoparticles, comprising an albumin coating and/or a rod shape, may additionally comprise a signal for release of the payload from the nanoparticle at a desired time or location. Such signals include, for example, contact between the nanoparticle and a target cell, tissue, organ or subject, or a change of an environmental parameter, such as the pH, ionic condition, temperature, pressure, and other physical or chemical changes surrounding the nanoparticle.

In one aspect, the present disclosure provides an immunogenic composition comprising an effective amount of nanoparticle that comprises an antigen or a hapten. A vaccine comprising the immunogenic composition is also provided. The vaccine composition disclosed herein may be administered prophylactically to a subject, i.e., a human, before infection with a pathogen, or may be therapeutically administered to subjects after infection with a pathogen. The term “vaccine” refers to a composition that is able to stimulate an immune response to a pathogen. Here, the term “immune response” includes either or both a humoral immune response and a cellular immune response.

The present disclosure further provides a medicament delivery system, and/or a pharmaceutical composition comprising the disclosed nanoparticles coated in albumin and/or having a rod-shape. Additionally, the nanoparticles may comprise a payload. Said pharmaceutical compositions comprise the provided nanoparticles in a pharmaceutically acceptable carrier or excipient for treating or preventing a disease or condition. The carrier typically includes a diluent, an excipient, a stabilizer, a preservative, and the like. Suitable examples of the diluent may include non-aqueous solvents such as propylene glycol, polyethylene glycol, vegetable oil such as olive oil and peanut oil, or aqueous solvents such as saline (for example, 0.8% saline), water (for example, 0.05 M phosphate buffer) containing a buffer medium, and the like, suitable examples of the excipient may include starch, glucose, lactose, sucrose, gelatin, malt, rice, flour, chalk, silica gel, sodium stearate, glycerol monostearate, talc, sodium chloride, anhydrous skimmed milk, glycerol, propylene, glycol, water, ethanol and the like, and suitable examples of the stabilizer may include carbohydrates such as sorbitol, mannitol, starch, sucrose, dextran, glutamate, and glucose, or proteins such as animal, vegetable or microbial proteins such as milk powder, serum albumin and casein.

The present disclosure further provides for the use of an effective amount of the pharmaceutical composition comprising the nanoparticles disclosed herein for the manufacture of a medicament for treating or preventing a disease or disorder in subject in need. Treatments, prevention, diagnosis and/or prognosis of any diseases, disorders, or physiological or pathological conditions, including, but not limited to, an infectious disease, a parasitic disease, a neoplasm, a disorder involving the immune mechanism, endocrine, nutritional, and metabolic diseases, inflammatory disease, diseases of the nervous, circulatory, respiratory, digestive, musculoskeletal or circulatory system, diseases of the skin and subcutaneous tissue, to name a few. In a specific embodiment, the nanoparticles disclosed herein, due to their efficient targeting to the lymphatic system including the lymph nodes, are particularly well suited for treatment of immune disorders and for stimulation of the immune system, e.g., for use as a vaccine.

In certain embodiments, the medicament delivery system and/or the pharmaceutical composition, in addition to the nanoparticles, further comprises one or more additional active ingredients and/or a medically or pharmaceutically acceptable carriers or excipients, that can be administered along with or in combination with the nanoparticles disclosed herein. In certain embodiments the medicant is a drug useful for treatment of immune disorders, inflammatory disorders, and cancers.

In one aspect, the disclosed nanoparticles, the medicament delivery system, or the pharmaceutical composition comprising the same are administered via any suitable administration route. For example, the nanoparticle, the medicament delivery system, or the pharmaceutical composition can be administered via an oral, nasal, inhalational, parental, intravenous, intraperitoneal, subcutaneous, intramuscular, intradermal, topical, or rectal route. Use of an effective amount of nanoparticles for the manufacture of a medicament for treating or preventing a disease or condition in a subject in need is also provided.

The present disclosure further provides a method for making the nanoparticles disclosed herein. Such methods, well known those of skill in the art include, for example, Chemical reduction, coprecipitation, seeding, microemulsion, inverse microemulsion, hydrothermal method, and sonic deposition. The rod-shaped nanoparticles may be generated using previously described mechanical stretching methods. For coating with albumin, the nanoparticles may be incubated with a solution comprising said albumin.

The present disclosure provides a kit that includes the nanoparticle compositions disclosed herein. In one specific aspect the kit further includes instructions for the treatment and/or prophylaxis of a disease or disorder. The nanoparticle compositions may, if desired, be presented in a pack or dispenser device which may contain one or more-unit dosage forms containing the nanoparticle compositions. The kit may be accompanied by instructions for administration to subjects, especially humans.

### BRIEF DESCRIPTION OF FIGURES

In order to better understand the subject matter that is disclosed herein and to exemplify how it may be carried out in practice, embodiments will now be described, by way of non-limiting example, with reference to the accompanying drawings. With specific reference to the drawings, it is stressed that the particulars shown are by way of example and for purposes of illustrative discussion of embodiments of the disclosure.

FIG. 1. Lymphatics expand from collapsed state due to interstitial flow (top). Lymphatics transport materials from peripheral tissues via capillaries and collecting vessels to the lymph nodes (center). Lymphatics transport materials across the vessel wall via paracellular and transcellular transport routes (bottom).

FIG. 2A-E. Schematic of Lymphatic Vessel Transport Properties FIG. 2A Lymphatic vascular system consists of (1) lymphatic capillaries, (2) collecting lymphatic vessels, (3) lymph nodes, and (4) the thoracic duct and right lymphatic trunk. Image adapted from (Aspelund). FIG. 2B Discontinuous basement membrane and button junctions (dotted lines) allow for lymphatic capillaries to absorb interstitial solutes, macromolecules, and immune cells. FIG. 2C Collecting vessels contain zipper-like junctions (continuous lines) and unidirectional valves. FIG. 2D Schematic of lymph node with multiple afferent and a single efferent vessel. FIG. 2E Lymphovenous valves.

FIG. 3A-E. Formulation of PEGylated Rod-Shaped Nanoparticles FIG. 3A Diameter of nanoparticles measured using DLS FIG. 3B Calculated Rf/D of nanoparticles. FIG. 3C Surface charge of nanoparticles measured via PALS FIG. 3D PDI of nanoparticles measured by DLS. FIG. 3E TEM images of nanoparticles after stretching and PEGylation protocol. Data presented as mean±SEM (*p<0.05) n=3-4.

FIG. 4A-D. Albumin Protein Corona on Nanoparticle Improves Nanoparticle Transport Across LECs FIG. 4A BCA protein quantification of nanoparticles in protein solutions FIG. 4B Transport efficiency of nanoparticles across hLECs pre-incubated in 10 mg/mL albumin FIG. 4C IF images of sectioned lymph nodes with nucleus and nanoparticles. FIG. 4D Quantification of IF Images. Data presented as mean±SEM (*p<0.05, #p,0.1) n=2-4

FIG. 5A-D. Interstitial Fluid Flow Improves Nanoparticle Transport Across Lymphatics FIG. 5A Schematic of lymphatic transport model FIG. 5B Transport efficiency over time of 100 nm fully PEGylated PSPEGRf/D-4.8 nanoparticles across hLECs under 10, 1, and 0 μm/s and fold increase of transport compared to static conditions. FIG. 5C IF images of hLECs with VE-Cadherin, nucleus, and nanoparticles stained. FIG. 5D MFI of IF images from lymphatic model. Data presented as mean±SEM (*p<0.05, #p<0.1) n=3-4.

FIG. 6A-F. Interstitial Fluid Flow Improves Rod-Shaped Nanoparticle Transport Across Lymphatics FIG. 6A Transport efficiency of nanoparticles across lymphatic transport model. FIG. 6B Transport efficiency of rod-shaped nanoparticles across lymphatic model in the presence of different transport inhibitors. FIG. 6C Transport efficiency of 100 nm spherical nanoparticles and rod-shaped nanoparticles across lymphatic transport model in the presence of flow and fold increase of transport compared to static conditions. FIG. 6D Transport efficiency of rod-shaped nanoparticles across lymphatic model in the presence of different transport inhibitors and interstitial flow. FIG. 6E MFI of IF images from lymphatic model. Data presented as mean FIG. 6F IF images of hLECs with VE-Cadherin stained, nucleus stained, and nanoparticles stained. Data as ±SEM (*p<0.05) n=3-4.

FIG. 7A-C. Rod Shaped Nanoparticles Reach Lymph Nodes More Efficiently Compared to similarly Sized Nanoparticles FIG. 7A IVIS imaging of C57Bl/6J after intradermal injection at 0 h and 8 h. FIG. 7B MFI of homogenized lymph nodes at 8 h. FIG. 7C IF images of sectioned lymph nodes with nucleus stained and nanoparticles stained. Data presented as mean±SEM (*p<0.05) n=3-4 mice

### DETAILED DESCRIPTION

The practice of the present disclosure will employ, unless otherwise indicated, conventional techniques of nanotechnology, nano-engineering, molecular biology (including recombinant techniques), microbiology, cell biology, biochemistry, immunology, and pharmacology, which are within the skill of the art. Unless defined otherwise, all technical and scientific terms used herein have the same meaning as is commonly understood by one of ordinary skill in the art to which this disclosure belongs.

The present disclosure provides novel nanoparticles, and methods of using and making thereof. More specifically, the provided nanoparticles comprise (i) an outer surface comprising albumin; and/or a rod-shaped nanoparticle. In a specific embodiment, the nanoparticle may further comprise a coating of polyethylene glycol (PEG). Nanoparticles that are rod-shaped and/or coated on the outer surface with albumin, have been shown herein to effectively cross lymphatic barriers, independent of ligand directed binding, and can therefor be used as an effective strategy to improve the efficacy and translatability of putative immunotherapies.

The term “nanoparticle” as used herein refers to nanostructure, particles, vesicles, or fragments thereof having at least one dimension (e.g., height, length, width, or diameter) of between about 1 nm and about 10 pm. For systemic use, an average diameter of about 50 nm to about 500 nm, or 100 nm to 250 nm may be preferred. In certain embodiments, the nanoparticles provided herein are biocompatible and/or biodegradable. The nanoparticles can be composed of organic materials or other materials and can alternatively be implemented with porous particles. The layer of nanoparticles can be implemented with nanoparticles in a monolayer or with a layer having agglomerations of nanoparticles.

As used herein, the nanoparticle consists of an albumin coating and/or a rod-shape. Rod-shaped as disclosed herein refers to an anisotropic nanoparticle with an aspect ratio of >1. In one embodiment the aspect ratio is >1.2. Regarding the albumin coating, the albumin may be derived from any animal source. In a specific embodiment, the albumin is derived from human. A range of different concentrations of albumin may be used for incubation and coating of the nanoparticles. Albumin concentration ranges can be between 500 mg/mL to 1 ng/ml. In an embodiment the albumin concentration is between 100 mg/mL and 100 ng/ml. In another embodiment, the concentration is between 50 mg/mL and 500 ng/mL. In yet another embodiment, the concentration is 10 mg/mL.

Examples of biocompatible polymers include polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In some cases, the nanoparticle is formed from a polyethylene glycol (PEG), poly(lactide-co-glycolide) (PLGA), polyglycolic acid, poly-beta-hydroxybutyrate, polyacrylic acid ester, or a combination thereof.

In a specific embodiment the nanoparticle is a nanoliposome. Such nanoliposomes may be composed of phospholipids such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-distearoyl-sn-glycero-3-phospho-(1′-rac-glycerol) (DSPG), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1,2-diolcoyl-sn-glycero-3-phospho-(1′-rac-glycerol) (DOPG), dipalmitoyl phosphatidylserine (DPPS), distearoyl phosphatidylserine (DSPS), dipalmitoyl phosphatidylinositol (DPPI), distearoyl phos phatidylinositol (DSPI), dipalmitoyl phosphatidic acid (DPPA), distearoyl phosphatidic acid (OSPA), 1,2-diacyl-3-trimethylammonium-propanes, (including but not limited to, dioleoyl (DOTAP), 1,2-dipalmitoyl-sn-glycero-3-phosphoethanolamine-N [methoxy(polyethylene glycol)-2000](DPPE-PEG2000), 1,2-distearoyl-sn-glycero-3-phosphoethanolamine-N-[methoxy(polyethylene glycol)-1000] (DSPE-PEG2000), and cholesterol.

The present disclosure further provides that the inventive nanoparticle, having an albumin coating and/or a rod-shape, comprises a payload that can be located in any place inside or on the surface of the nanoparticle. In certain embodiments, the payload comprises one or more therapeutic agents, prophylactic agents, diagnostic agents, or a combination thereof. Examples of therapeutic agents include, but are not limited to, an antibiotic, an antimicrobial, a growth factor, a chemotherapeutic agent, an anti-inflammatory, immunomodulators, metastasis inhibitors immunosuppressants including but not limited to adaptive immunosuppressants, cancer therapeutics, vaccines, or a combination thereof.

In another aspect, the payload includes a toxin and may be used to target cell death to a particular cell or tissue target. As used herein, the “toxin” refers to a toxic material or product of plants, animals, microorganisms (including, but not limited to, bacteria, virus, fungi, rickettsiae or protozoa), or infectious substances, or a recombinant or synthesized molecule that targets cell death. In certain embodiments, the payload is a metallic particle, a polymeric particle, a dendrimer particle, or an inorganic particle.

In a specific embodiment, the payload comprises an antigen or hapten for stimulation of a protective immune response. The targeting of the provided nanoparticles to the lymphatic system, including the lymph nodes, provides an efficient means for stimulation of a desired immune response. In an embodiment, the antigen or hapten is derived from the surface of a tumor cell and may be used to stimulate an immune response against the tumor cells. In another embodiment, the antigen or hapten, comprises an allergen or self-antibody for use in development of tolerance to said allergen or self-antibody. In some embodiments, the payload is coated on the nanoparticle using a crosslinking agent. In some embodiments, the payload is adsorbed onto the nanoparticle surface. In some embodiments, the payload is adsorbed onto the nanoparticle surface followed by covalent crosslinking of the payload to the nanoparticle surface using a crosslinking agent.

Crosslinking agents suitable for crosslinking the proteins to produce the nanoparticle, or to coat the proteins on the nanoparticle are known in the art, and include those selected from the group consisting of formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfhydryl addition or disulfide reduction, carbohydrate reactive crosslinkers, carboxyl reactive crosslinkers, photoreactive crosslinkers, cleavable crosslinkers, AEDP, APG, BASED, BM(PEO)3, BM(PEO)4, BMB, BMDB, BMH, BMOE, BS3, BSOCOES, DFDNB, DMA, DMP, DMS, DPDPB, DSG, DSP, DSS, DST, DTBP, DTME, DTSSP, EGS, HBVS, sulfo-BSOCOES, Sulfo-DST, and Sulfo-EGS.

The provided nanoparticles, comprising an albumin coating and/or a rod shape, may additionally comprise a signal for release of the payload from the nanoparticle at a desired time or location. Such signals include, for example, contact between the nanoparticle and a target cell, tissue, organ or subject, or a change of an environmental parameter, such as the pH, ionic condition, temperature, pressure, and other physical or chemical changes surrounding the nanoparticle.

The present disclosure further provides a medicament delivery system, and/or a pharmaceutical composition comprising the disclosed nanoparticles coated in albumin and/or having a rod-shape. Additionally, as provided above, the nanoparticles may comprise a payload. In one aspect, the present disclosure provides nanoparticle immunogenic compositions comprising an effective amount of a nanoparticle that is associated with an antigen or a hapten. A vaccine comprising the immunogenic composition is also provided.

Pharmaceutical compositions of embodiments comprise a therapeutically effective amount of nanoparticles dissolved or dispersed in a pharmaceutically acceptable carrier. The preparation of a pharmaceutical composition that contains the nanoparticles and optionally an additional active ingredient will be known to those of skill in the art in light of the present disclosure, as exemplified by Remington's Pharmaceutical Sciences, 18th Ed. Mack Printing Company, 1990. For human administration, it will be understood that preparations should meet sterility, pyrogenicity, general safety and purity standards as required by FDA Office of Biological Standards or corresponding authorities in other countries. Preferred compositions are lyophilized formulations or aqueous solutions.

As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in therapeutic or pharmaceutical compositions is contemplated.

The composition may comprise different types of carriers depending on whether it is to be administered in solid, liquid or aerosol form, and whether it needs to be sterile for such routes of administration as injection. Nanoparticles of certain embodiments (and any additional therapeutic agent) can be administered by any method, or any combination of methods as would be known to one of ordinary skill in the art (see, for example, Remington's Pharmaceutical Sciences, 18th Ed. Mack Printing Company, 1990, incorporated herein by reference). Parenteral administration, in particular intravenous injection, may be used for administering the nanoparticles of certain embodiments. Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

For immunogenic compositions, said compositions further comprise an immunogenic adjuvant or immunopotentiator. As used herein, the “immunogenic adjuvant” is a substance or composition which can induce and/or enhance an immune response against an antigen. As used the “immunopotentiator” refers to an agent that upon inoculation enhances the immune response. Exemplary immunogenic adjuvant can be Freund's complete adjuvant which is a mixture of light mineral oil, Arlacel detergent, and inactivated Mycobacterium tuberculosis bacilli. Exemplary immunopotentiator includes Bacille Calmette-Guerin (BCG), Corynebacterium parvum, Brucella abortus extract, glucan, levamisole, tilorone, an enzyme and a non-virulent virus.

The provided nanoparticles may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as, for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

Pharmaceutical compositions comprising the disclosed nanoparticles may be manufactured by means of conventional mixing, dissolving, emulsifying, encapsulating, entrapping or lyophilizing processes. Pharmaceutical compositions may be formulated in conventional manners using one or more physiologically acceptable carriers, diluents, excipients or auxiliaries which facilitate processing of the proteins into preparations that can be used pharmaceutically. Proper formulation is dependent upon the route of administration chosen.

In certain embodiments, the medicament delivery system and/or the pharmaceutical composition, in addition to the nanoparticles, further comprises one or more additional active ingredients and/or a medically or pharmaceutically acceptable carriers or excipient, that can be administered along with or in combination with the nanoparticles disclosed herein. In certain embodiments the medicant is a drug useful for treatment of immune disorders, inflammatory disorders, and cancers.

The present disclosure further provides the use of a therapeutically effective amount of the pharmaceutical composition comprising the nanoparticles disclosed herein for the manufacture of a medicament for treating or preventing a disease or disorder in a subject in need. Treatments, prevention, diagnosis and/or prognosis of any diseases, disorders, or physiological or pathological conditions, including, but not limited to, an infectious disease, a parasitic disease, a neoplasm, a disorder involving the immune mechanism, endocrine, nutritional, and metabolic diseases, inflammatory disease, diseases of the nervous, circulatory, respiratory, digestive, musculoskeletal or circulatory system, diseases of the skin and subcutaneous tissue, to name a few. In certain embodiments, the nanoparticle compositions provided herein may be used as vaccines for treating or preventing infectious diseases caused by pathogenic microorganisms, such as bacteria, viruses, parasites or fungi. In other embodiments, the inventive compositions are used for treating or preventing cancer or a neoplasm condition.

As used herein, a subject in need refers to an animal, a non-human mammal, or a human. In a specific embodiment, the nanoparticles disclosed herein, due to their efficient targeting to the lymphatic system including the lymph nodes, are particularly well suited for treatment of immune disorders and for stimulation of the immune system, e.g., for use as a vaccine.

The terms “treat/treating/treatment” and “prevent/preventing/prevention” as used herein, refers to eliciting the desired biological response, i.e., a therapeutic and prophylactic effect, respectively. In accordance with the present disclosure, the therapeutic effect includes one or more of a decrease/reduction in the severity of the disease, a decrease/reduction in symptoms and disease related effects, an amelioration of symptoms and disease-related effects, and an increased survival time of the affected host, following administration of the nanoparticle composition.

As used herein, the term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition. The determination of a therapeutically effective amount is well within the capabilities of those skilled in the art, especially in light of the detailed disclosure provided herein.

In one aspect, the disclosed nanoparticles, the medicament delivery system, or the pharmaceutical composition comprising the same are administered via any suitable administration route. For example, the nanoparticle, the medicament delivery system, or the pharmaceutical composition can be administered via an oral, nasal, inhalational, parental, intravenous, intraperitoneal, subcutaneous, intramuscular, intradermal, topical, or rectal route. Use of an effective amount of nanoparticles for the manufacture of a medicament for treating or preventing a disease or condition in a subject in need is also provided.

The attending physician for patients treated with the disclosed nanoparticles, the medicament delivery system, or the pharmaceutical composition comprising the same would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

In another aspect, the present disclosure provides a method of use of an immunogenic composition comprising the provided nanoparticles, with an albumin coating and/or a rod-shape and an antigen or hapten, for eliciting an immune response to an antigen or hapten in a subject in need of such elicitation, and method of use of the nanoparticle as a vaccine comprising the immunogenic composition for protecting a subject against the antigen or hapten.

As used herein the term vaccine refers to a composition capable of eliciting in a patient a beneficial active or passive immune response to a specific antigen. While protective immunity may be desired, it is understood that various levels of temporal immune response can be beneficial. In certain embodiments, the immune response is T-cell or B-cell mediated immune response. Accordingly, use of an effective amount of the nanoparticle for the manufacture of the immunogenic composition against an antigen or hapten, and use of an effective amount of the immunogenic composition for the manufacture of a vaccine for protecting a subject against the antigen or hapten, are provided.

The present disclosure is directed to methods of generating an immune response in a subject to a vaccine formulation, e.g., said formulation comprising the nanoparticles provided herein containing an antigen or hapten. More specifically, the present disclosure is directed to methods of generating an immune response in a subject, comprising administering an immunologically effective amount of a vaccine formulation of the present disclosure to a subject, thereby generating an immune response in a subject. In each of the methods of generating an immune response of the present disclosure, the immune response is preferably a protective immune response.

An “immunologically effective amount” of a vaccine formulation is one that is sufficient to induce an immune response to vaccine components in the subject to which the vaccine formulation is administered. A “protective immune response” is one that confers on the subject to which the vaccine formulation is administered protective immunity against the pathogen from which the antigens or haptens of the formulation were obtained. The protective immunity may be partial or complete immunity.

The vaccine formulations of the present disclosure may also be used in methods of inhibiting a pathogenic infection in a subject. Such methods comprise administering a therapeutically effective amount of a vaccine formulation of the present disclosure to a subject at risk of developing an infection, thereby inhibiting an infection in a subject.

A “therapeutically effective amount” of a vaccine formulation is one that is sufficient to provide at least some reduction in the symptoms of a pathogen infection in a subject to which the vaccine formulation is administered.

As used herein, the terms “inhibit”, “inhibiting” and “inhibition” have their ordinary and customary meanings and include one or more of inhibiting the pathogen. Such inhibition is an inhibition of about 1% to about 100% versus a subject to which the vaccine formulation has not been administered. As used herein, the inhibition lasts at least a period of days, weeks, months or years upon completing of the dosing schedule. Preferably the inhibition is for the lifespan of the subject.

The present disclosure is also directed to methods for providing prophylaxis of a pathogen infection in a subject using the vaccine formulations of the present disclosure. In one embodiment, the present disclosure is directed to methods for providing prophylaxis of a pathogen infection in a subject, comprising administering a therapeutically effective amount of a vaccine formulation of the present disclosure to a subject having a pathogen infection, thereby providing prophylaxis of the infection in a subject.

As used herein, “prophylaxis” includes inhibiting the development of a productive or progressive infection by a pathogen in a subject, where the prophylaxis lasts at least 1, 5, 10, 15, 20, 25, 30, 35, 40, 45, 50, 55, 60, or more days after administration of a vaccine formulation the present disclosure. Inhibition against development of a productive or progressive infection by the pathogen means that the severity of an infection in a subject is reduced by about 1% to about 100% versus a subject to which a vaccine formulation of the present disclosure has not been administered.

In the methods of the present disclosure the vaccine formulations are administered in a pharmaceutically acceptable form and in substantially non-toxic quantities. The vaccine formulations may be administered to a subject using different dosing schedules, depending on the particular use to which the formulations are put (e.g., administration to the subject pre- or post-exposure to a pathogen), the age and size of the subject, and the general health of the subject, to name only a few factors to be considered.

The present disclosure further provides a method for making the nanoparticles disclosed herein. Such methods, well known to those of skill in the art include, for example, Chemical reduction, coprecipitation, seeding, microemulsion, inverse microemulsion, hydrothermal method, and sonic deposition. The rod-shaped nanoparticles may be generated using previously described mechanical stretching methods. For coating with albumin, the nanoparticles may be incubated with a solution comprising said albumin.

In another aspect of the embodiment, an article of manufacture (e.g., a kit) containing materials useful for the treatment or prevention of diseases or disorders treatable by administration of the nanoparticles, as described above, is provided. In an embodiment, the kit comprises the necessary components of a vaccine formulation that elicits an immune response to and instructions for its use is also provided herein.

The article of manufacture comprises a container and a label or package insert on or associated with the container. Suitable containers include, for example, bottles, vials, syringes, IV solution bags, etc. The containers may be formed from a variety of materials such as glass or plastic. The article of manufacture may comprise (a) a first container with a composition contained therein, wherein the composition comprises a nanoparticle comprising a coating of albumin and/or a rod-shape; and (b) a second container with a composition contained therein, wherein the composition comprises a further therapeutic agent.

Kits in certain embodiments may further comprise a package insert indicating that the compositions can be used to treat a particular condition. Alternatively, or additionally, the kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

All documents, papers and published materials referenced herein, including books, journal articles, manuals, patent applications, published patent applications and patents, are expressly incorporated herein by reference in their entireties.

### Examples

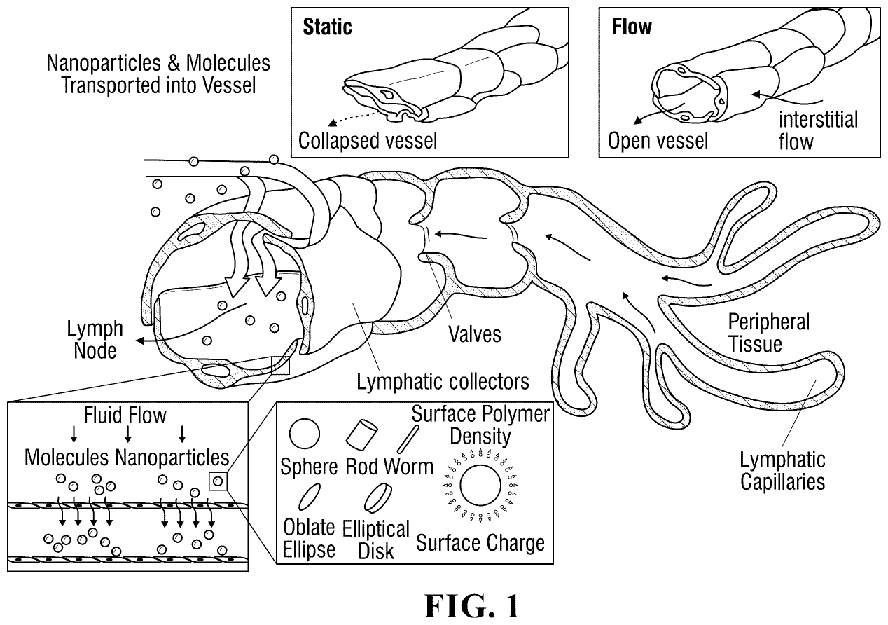

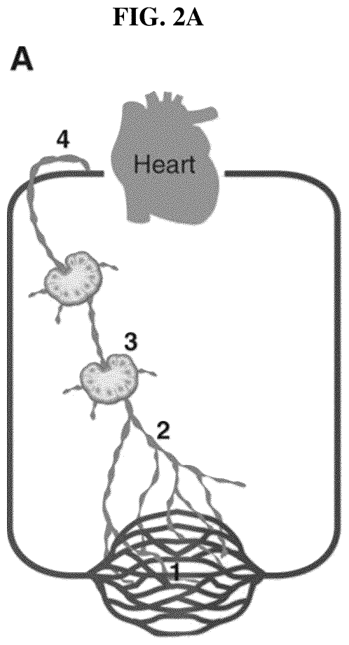

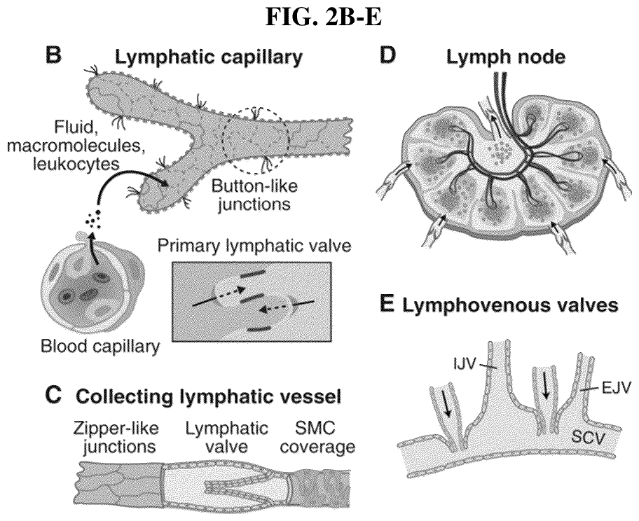

Lymphatics exist throughout the body and are appreciated for the transport of materials to the lymph nodes where immune responses are formed. (FIG. 1 and FIG. 2) This has caused lymphatic vessels to become potential drug delivery targets to transport immune modulatory therapies to the LNs without requiring direct injections. Delivering immunotherapies, including vaccines, to the LNs has been shown to potentiate their therapeutic effects, particularly crucial as efficacy of many immunotherapies still requires improvement. Recent studies have demonstrated that nanoparticles between 10-250 nm in diameter are transported preferentially via lymphatic vessels from peripheral tissues to LNs, highlighting that the transport functions of lymphatics can be taken advantage of for drug delivery. Additionally, studies have identified the key nanoparticle surface chemistry required for transport across lymphatics, and the mechanisms governing lymphatic transport: nanoparticles with PEG in a dense brush conformation on the surface of nanoparticles utilize both transcytosis and paracellular transport mechanisms. Interestingly, it has been observed that densely PEGylated nanoparticles do not rely on macropinocytosis to cross lymphatic barriers. This change in transport mechanism could be the result of changes in the protein corona formed on nanoparticles in a formulation dependent manner.

In addition to protein corona formation, other key physiological phenomena like transmural flow have been shown to affect lymphatic physiology and transport, should be considered to better recapitulate in-vivo conditions within transport models. While key formulation parameters have been identified including size, charge, and surface chemistry that facilitate lymphatic transport, there remains a need to understand how the lymphatic environment in-vivo can affect lymphatic transport, critically, 1) the formation of the protein corona on nanoparticle surface post injection, and 2) the effect that physiological interstitial flow has on lymphatic transport.

When nanoparticles and other foreign materials enter biological environments, the surrounding molecules and proteins quickly adhere onto the surface creating a protein corona. This adsorption has been demonstrated to influence drug delivery applications of nanoparticles. Some studies have demonstrated that the formation of protein corona on the surface of nanoparticles can hinder nanoparticle transport across endothelial and epithelial barriers by blocking nanoparticle interactions with the cell membrane. However, other studies have indicated that the formation of protein corona on the surface of nanoparticles can improve transport and uptake of nanoparticles across, especially if protein components are ligands for corresponding receptors on cells of interest. This role of the protein corona with regards to improved nanoparticle transport is of special interest with regards to lymphatics. Albumin, one of the most ubiquitous proteins found in the body, and a common component of the protein adsorbed on foreign material, has been utilized to improve lymphatic transport.

Another key consideration for drug delivery into lymphatics is the interstitial fluid flow that continuously drives fluid and material into initial lymphatic capillaries, and on through collecting lymphatic vessels into lymph nodes. Indeed, this interstitial flow into lymphatics is considered to be a major contributor to the transport of material into lymphatics. It has also been found that the presence of interstitial flow is key in facilitating cellular transport mechanisms within the lymphatics, suggesting that investigating the transport of nanoparticles in lymphatics under standard conditions does not fully recapitulate the mechanisms present in-vivo. When considering the importance of flow for lymphatic drug delivery, an emerging nanoparticle formulation parameter to consider is shape. Studies have demonstrated that rod-shaped nanoparticles can align with flow and have improved circulating times compared to traditional spherical nanoparticles. Another draw of using rod-shaped nanoparticles is that they have been found to better diffuse through extracellular matrix and mucus barriers. This further makes rod shaped nanoparticles an attractive candidate for lymphatic delivery as the extracellular matrix is one of the key barriers for entering and traversing the lymphatics after intradermal or intraperitoneal administration of nanoparticles.

As described herein, the effects of protein corona formation, interstitial flow, and nanoparticle shape on the delivery of nanoparticles into lymphatic vessels was studied, as well as how these parameters affect the transport mechanisms governing transport into the lymphatic vessels. A previously established model for lymphatic transport was used incorporating simulated transmural fluid flow to examine how flow promotes transport in a shape and mechanism dependent manner. To explore the effect of protein corona, polyethylene glycol (PEG) coated nanoparticles were used that had been previously identified to effectively cross lymphatic barriers and reach lymph nodes and examined how parameters like PEG density effected corona formation and subsequent transport. To generate rod shaped nanoparticles previously established stretching methods were used and the resultant rod-shaped nanoparticles were then used in vitro and in vivo lymphatic methods.

### Materials and Methods

Nanoparticle formulation. Rod shaped nanoparticles were generated using a previously described mechanical stretching method. 20 mL of 5% w/v solution of polyvinyl alcohol (PVA) was mixed using a magnetic stir bar and heated at 130° C. for at least 1 h until fully dissolved. Once dissolved, 100 nm, fluorescent carboxyl-coated polystyrene nanoparticles (ThermoFisher) were mixed into the PVA solution 0.1% w/v. To plasticize the film, 320 μL of glycerol was also added to the PVA/nanoparticle solution. The solution was then poured into a 12×12 cm mold and left to dry overnight uncovered. Once the film fully dried, 9×5 cm films were cut from the film and submerged in toluene for three hours to liquefy the nanoparticles. The films were loaded on an Arduino-controlled mechanical stretcher and stretched to up to 2 times the size of the marked grid and left to dry overnight. The film was cut along the grid marks and any non-uniformly stretched portions were discarded. The remaining film sections were submerged in isopropanol to remove any residual toluene. After 24 h, the film sections were removed and then put into a 30% isopropanol solution, which was then mixed and heated at 130° C. for an hour until the film had fully dissolved. The dissolved films were centrifuged for 30 min at 26,000×g, and the supernatant was discarded. The pellet was re-suspended in 30% isopropanol at 130° C. and after 1 h, centrifuged at 26,000×g. This process of PVA film dissolution and washing was repeated three times. To separate the rods from the spheres, solutions of stretched nanoparticles were washed at 12,000×g for 100 nm for 20 min. Stretched nanoparticles were stored in DI H2O.

To PEGylate the nanoparticles, 40 nm, 100 nm, 100 nm stretched, or 200 nm fluorescent carboxyl (COOH)-modified PS nanoparticles (Thermo Fisher Scientific, F8801) were covalently modified with 5 kDa MW methoxy-PEG-amine (NH2) (Creative PEGworks), as previously described Briefly, PS—COOH particles were suspended at 0.1% w/v in 200 mM borate buffer (pH=8.2). 350 μM PEG was conjugated to nanoparticles using 7 mM N-Hydroxysulfosuccinimide (NHS) (Sigma) and 0.02 mM 1-Ethyl-3-(3-dimethylaminopropyl) carbodiimide (EDC) (Invitrogen). The reaction was allowed to proceed on a rotary incubator at room temperature for at least 4 hours. Nanoparticles were collected using 100 k MWCO centrifugal filters (Amicon Ultra; Millipore) and washed with deionized (DI) water. Nanoparticles were resuspended at 1% w/v in DI water and stored at 4° C.

Nanoparticle characterization. Dynamic light scattering (DLS) was used to measure the hydrodynamic diameter and polydispersity index (PDI) of nanoparticles. Phase analysis light scattering (PALS) was used for measuring ζ-potential (NanoBrook Omni). Measurements were performed using a scattering angle of 90° at 25° C. Measurements were based on intensity of reflected light from scattered particles.

Transmission Electron Microscopy. Stretched nanoparticles were diluted 100× in ultrapure H2O and 25 μL was placed on a square of Parafilm. A PELCO® copper mesh grid (400 mesh Cu, Ted Pella) was inverted and placed on this droplet for 30 s. After, the grid was washed 4 times for 30 s by placing on a 25 μL droplet of ultrapure H2O. After drying, the grid was imaged using transmission electron microscopy (JEM-2100, 200 kV, JEOL Ltd).

PEG density characterization. PEG density was determined using previously published methods. Briefly, 5 kDa PEG-NH2 (Creative PEGworks) conjugated to fluorescein isothiocyanate (FITC) was conjugated to fluorescent (AlexaFluor®555) 100 nm carboxyl-modified nanoparticles. A FITC-PEG-NH2 standard curve was generated in DI water to calculate the PEG amount on the nanoparticle surface using a plate reader (Tecan Spark Multimode Microplate Reader). From these measurements, PEG grafting distance (D) and PEG density were estimated using the Flory radius of PEG (Rf). The Flory radius of a polymer chain is defined as Rf˜αN3/5, where N is the degree of polymerization, and α is the effective monomer length. An unconstrained 5 kDa PEG chain has a Rf of 5.4 nm and occupies 22.7 nm2. PEG density and conformation can be correlated to the ratio of Rf/D, with Rf/D<1-1.5 yielding a mushroom conformation, 1-1.5<Rf/D>4 yielding a brush conformation, and Rf/D>4 yielding a dense brush conformation.

Lymphatic transport model. Nanoparticle transport across LECs was assessed using an established in vitro model that recapitulates in vivo lymphatic transport. Briefly, primary human dermal LECs (hLECs, Promocell C-12217) were seeded on 1.0 μm pore size, 12 mm transwell inserts (Falcon) at 200,000 cells/cm2 and cultured in EGM2 (Lonza) at 37° C. and 5% CO2 for 48 h. Cells were pretreated with 1 μm/s transmural flow to simulate the tissue microenvironment. For experiments examining the effect of flow on transport, 1 μm/s or 0.5 μm/s flow rates were maintained throughout experiment by generating a fluid head within the transwell as previously described. hLECs were treated with 1% w/v nanoparticles on the apical side and the basolateral compartment was sampled for up to 24 h. Fluorescence intensity was measured using a plate reader (Tecan) and nanoparticles transported was calculated using a standard curve. Transport experiments were performed in EGM2 without growth factors to avoid the confounding effects of growth factors. To probe the transport mechanism the following transport inhibitors were used: 100 nM Adrenomedullin (Abcam ab276417), 62.5 μM Dynasore (Sigma D7693), or 62.5 μM Amiloride (Sigma A7410). Transport inhibitors were applied for 2 hours prior to the introduction of nanoparticles. To examine the effect of albumin protein corona formation on lymphatic transport, 100 nm PEGylated nanoparticles were incubated in 10 mg/mL albumin for 30 min. Nanoparticles were collected and separated from protein solution via centrifugation at 17,000×g for 20 minutes and washing with DI water twice before pellet was collected before being administered to the transport model at 1% w/v.

Immunofluorescence staining. Cells were fixed in 2% PFA for 15 minutes and incubated with mouse anti-human VE-Cadherin (BD Sciences) at 4° C. overnight. Secondary antibodies conjugated to Alexa Fluor® 488 or 647 were used for detection (Thermo Fisher). Slides were mounted using DAPI (4′,6-diamidino-2-phenylindole)-containing Vectashield (Vector Laboratories Inc., Burlingame, CA) and imaged using a Zeiss Axio Observer. Image processing was performed using FIJI (NIH).

C57Bl/6J lymphatic delivery model. 10 μL of 5 mg/mL, fluorescently labeled nanoparticles was intradermally administered to female C57Bl/6J mice (8-12 weeks old) in their forelimbs. Fluorescence intensity was measured using IVIS Spectrum Fluorescent & Chemiluminescent Imaging System (Caliper Life Sciences) over a 12 h time period. Distance of nanoparticle transport was calculated from centroid of injection site to maximally distant pixel of fluorescence signal using ImageJ. This pixel length was then converted to centimeters. Mice were anesthetized with isoflurane prior to nanoparticle injection and during imaging. Mice were euthanized after the final time point (8 hr). Draining LNs were collected and homogenized to quantify the fluorescence signal from nanoparticles using a plate reader (Tecan). LNs were also fixed in 4% PFA for 6 hours and treated with a sucrose gradient. Tissues were then embedded within OCT (ThermoFisher), sectioned, and stained for FITC-B220 (BioLegend). Slides were mounted using DAPI (4′,6-diamidino-2-phenylindole)-containing Vectashield (Vector Laboratories Inc., Burlingame, CA) and imaged using a Zeiss Axio Observer. Image processing was performed using FIJI (NIH). All procedures were approved by the University of Maryland, College Park IACUC.

Protein Corona Analysis. Nanoparticles were incubated in fetal bovine serum or 10 mg/mL albumin for 30 min. Nanoparticles were collected and separated from protein solution via centrifugation at 17,000×g for 20 minutes and washed with DI water twice before pellet was collected. The amount of protein on the nanoparticle surface was quantified using Pierce micro BCA assay kit (ThermoFisher). Absorbance at 562 nm was measured using a plate reader (Tecan Spark Multimode Microplate Reader).

Statistics. Group analysis was performed using a 2-way ANOVA, followed by Tukey's post-test. Unpaired Student's t-test was used to examine differences between only two groups. A value of p<0.05 was considered significant (GraphPad). All data is presented as mean±standard error of the mean (SEM).

### Results

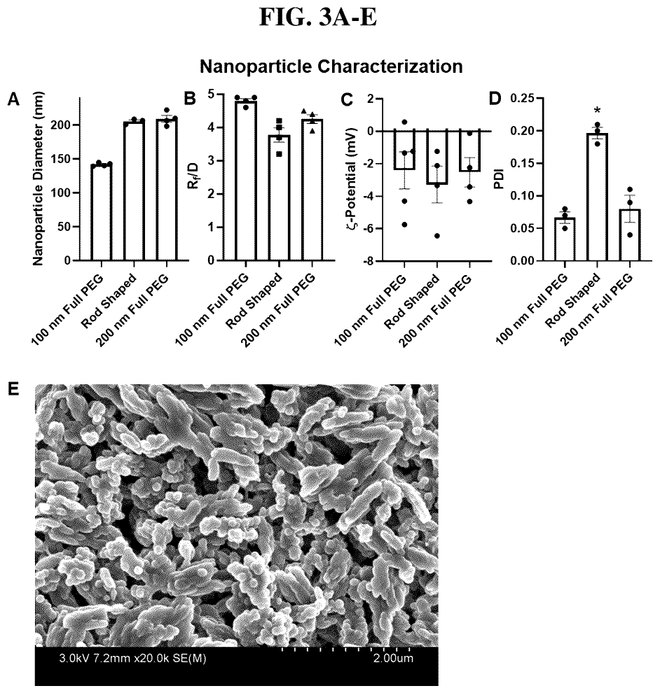

Protein Corona Forms Rapidly on PEGylated Nanoparticles and Improves Transport Across Lymphatics. Shape has been shown to affect how nanoparticles interact with surrounding tissues and cells. In this study PEGylated nanoparticles were used that have been previously shown to improve transport across lymphatic vessels and reach lymph nodes. Using the mechanical stretching technique, rod-shaped nanoparticles were generated. When stretched and PEGylated 100 nm nanoparticles increased in size from 142 nm to 201 nm, making the rod-shaped nanoparticles closer in size to the 200 nm PEGylated nanoparticles (198 nm) (FIG. 3A). When the Rf/D of the nanoparticles was calculated, all formulations and shapes had PEG in dense brush conformation (3.5-4.5) (FIG. 3B). When the surface charge of the fully PEGylated spherical nanoparticles and rod-shaped nanoparticles were measured, they were near neutral (>−5 mV), suggesting dense PEG coverage in conjunction with the Rf/D calculations (FIG. 3C). PDI of the nanoparticles measured via DLS indicated that the rod-shaped nanoparticles had a slightly non-uniform distribution of sizes with a PDI>0.2. This higher PDI is likely due to the anisotropic shape of the nanoparticles as well as the fact that the stretching process is not completely efficient: imaging via electron microscopy reveals a heterogenous sample containing both rods and spheres. This is likely due to the fact that the rod-shaped nanoparticles are anisotropic and the stretching protocol is not completely efficient (FIG. 3D-E).

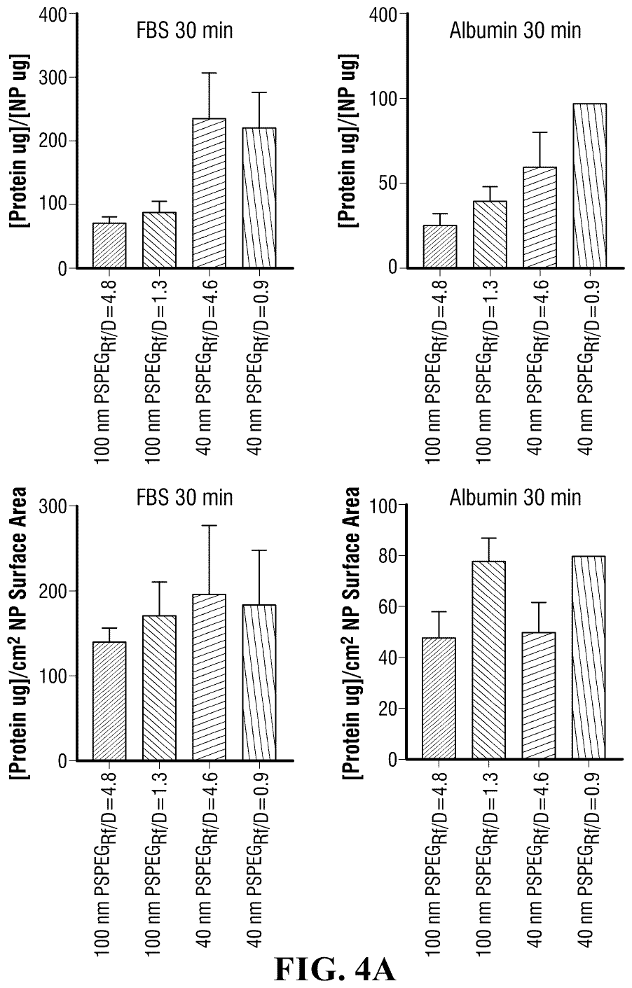

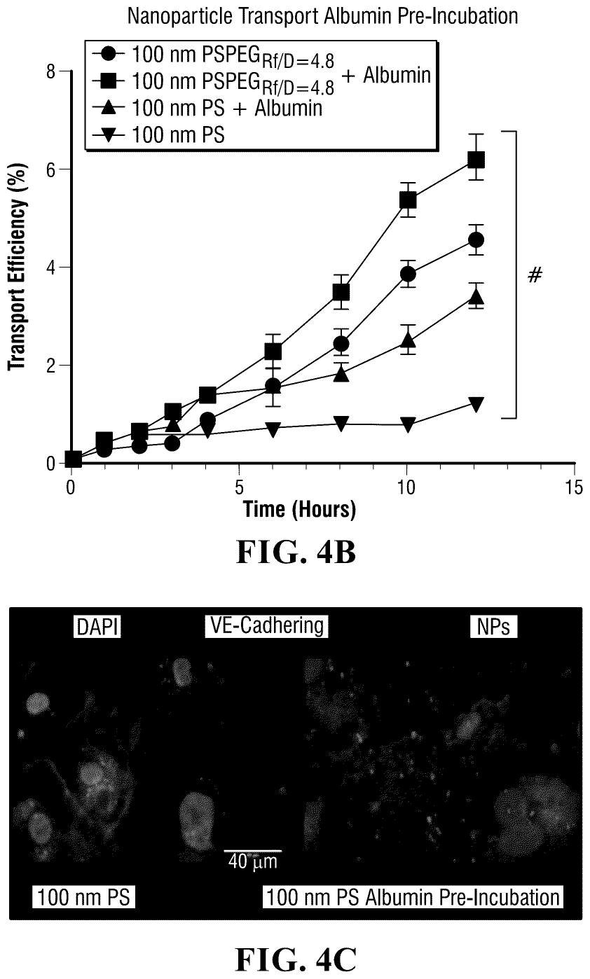

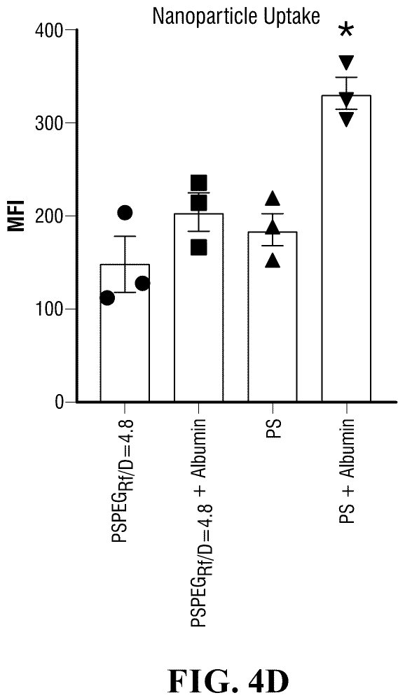

As a preliminary way to understand the formation of the protein corona on the surface of nanoparticles, and how this effects nanoparticle transport into lymphatics 40 nm and 100 nm nanoparticles were incubated with a dense coating of PEG (full PEG) and sparse coating of PEG (0.1PEG) n a 10 mg/mL albumin solution and FBS. It was found that between formulations, the amount of protein adsorbing to the surface of the nanoparticles remained consistent. In this preliminary experiment, it was observed that 40 nm PSPEGRf/D=0.9 nanoparticles have measurably more FBS protein on the surface compared to the fully PEGylated nanoparticles. (FIG. 4A). It was then examined if the addition of protein on the surface of the nanoparticles improved transport across LEC barriers using the transwell based model. Particularly, the role of albumin was examined, an extremely abundant protein that is known to be actively transported into lymphatics and is known to be a major protein corona component. The addition of albumin correlated with an increase in transport efficiency for both PEGylated 100 nm nanoparticles, and non-PEGylated 100 nm polystyrene nanoparticles. Indeed, the pre-incubation of the polystyrene nanoparticles resulted in a significant increase in transport efficiency, becoming comparable to the efficiently transported 100 nm PEGylated nanoparticles (FIG. 4B). When examining IF images of the LEC model, it was observed that there was an increase in the polystyrene nanoparticle signal when they are pre-incubated in albumin (FIG. 4C-D).

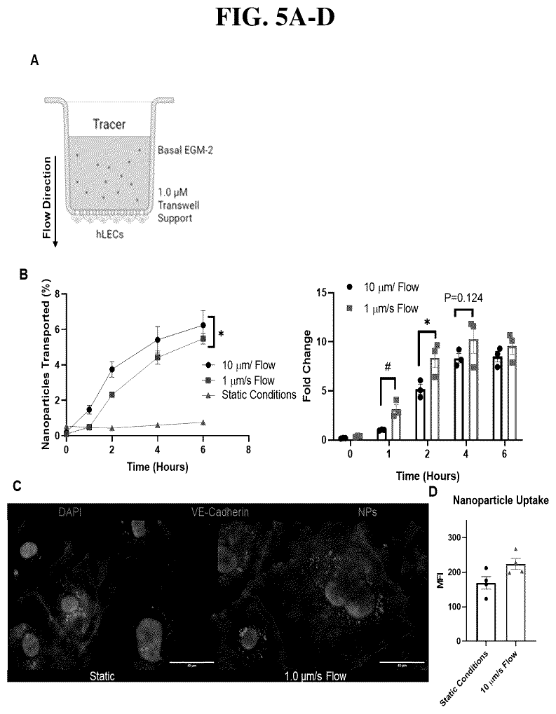

Interstitial Fluid Flow Improves Nanoparticle Transport Across Lymphatics. The fully PEGylated PSPEGRf/D=4.8 100 nm spherical nanoparticles were administered to a previously described in vitro transendothelial transport model of primary human LECs was cultured on the bottom of a collagen-coated transwell (FIG. 5A) to simulate transport from the interstitium into the lymphatic vessel. When looking at spherical PSPEGRf/D=4.8 nanoparticle transport in the presence of modeled transmural flow, it was observed that the presence of any transmural flow increased transport efficiency at least 5-fold after six hours (FIG. 5B). After IF imaging of the LEC model, it was observed that LECs treated with flow trended towards internalized higher amounts of fluorescent nanoparticles (not significant) (FIG. 5C-D).

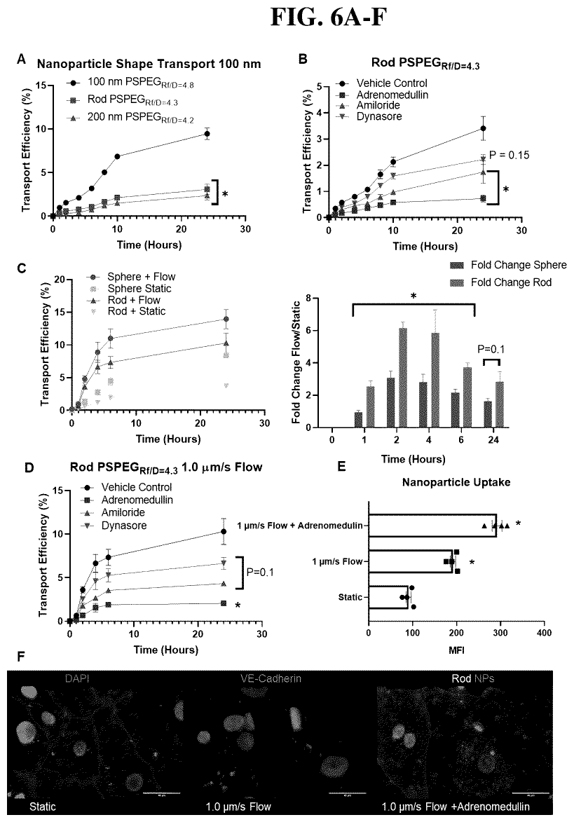

The fully PEGylated 100 nm PSPEGRf/D=4.8 spherical nanoparticles, the rod shaped PSPEGRf/D=4.3 nanoparticles, and the 200 nm fully PEGylated PSPEGRf/D=4.2 nanoparticles were administered to the lymphatic transport model to examine how shape effects the transport efficiency across lymphatics. It was observed that rod shaped nanoparticles and 200 nm nanoparticles were transported similarly while the 100 nm nanoparticles exhibited significantly higher transport efficiency (FIG. 6A). It was observed that when micropinocytosis was inhibited with Dynasore, and when macropinocytosis was inhibited with amiloride, there was a significant decrease in transport efficiency of rod PSPEGRf/D=4.3 nanoparticles. Importantly, when paracellular transport was inhibited with adrenomedullin, closing the spaces between cells, the greatest decrease in transport efficiency was observed (FIG. 6B). Simulated transmural flow drives the transport of fluid into lymphatic vessels in vivo and has been shown to affect LEC permeability. Experiments were designed to probe how the presence of physiologically relevant transmural flow affected the transport of rod-shaped nanoparticles across lymphatic barriers. Interestingly, in the presence of model flow it was observed that rod shaped nanoparticles were transported up to 6-fold more efficiently compared to static conditions. This is a marked increase compared to the 3-fold increase observed with respect to the spherical nanoparticles in the presence of transmural flow (FIG. 6C). When the transport mechanisms of rod-shaped nanoparticles were observed under flow conditions, similar trends to the static conditions were observed where inhibition of paracellular transport through the introduction of adrenomedullin caused the greatest reduction in transport efficiency (FIG. 6D). IF imaging of LEC models indicated that there was intracellular uptake of the rod-shaped nanoparticles. Notably, when paracellular transport was inhibited, an increase in nanoparticle signal within the cells was observed. (FIG. 6D-E).

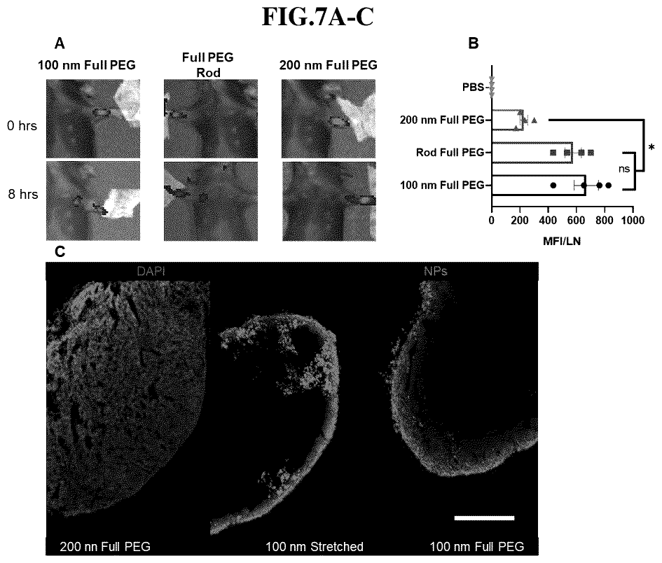

Rod Shaped Nanoparticles Reach Lymph Nodes More Efficiently Compared to similarly Sized Nanoparticles. To confirm the observed transport trends on lymphatic transport in-vitro with delivery to lymph nodes, the spherical 100 nm and 200 nm nanoparticles, as well as the rod-shaped nanoparticles, were intradermally injected. IVIS imaging of the fluorescent nanoparticles indicated that the 100 nm nanoparticles and the rod-shaped nanoparticles are present in lymph nodes after 8 hrs (FIG. 7A). This time frame is indicative of lymphatic vessel mediated transport as opposed to cell migration mediated transport which typically requires periods of over 12 hrs. Lymph nodes were collected after 8 hours and homogenized for fluorescence measurement. It was found that the fluorescence signal of the rod-shaped nanoparticles was similar to that of the spherical 100 nm nanoparticles, despite the rod nanoparticles being closer in size to the 200 nm spherical nanoparticles (FIG. 7B). Furthermore, when examining the localization of the rod-shaped nanoparticles in collected lymph nodes, it was observed that there seems to be nanoparticle signal in the cortex of the lymph node (FIG. 7C)

The experiments described herein sought to determine how nanoparticle shape affects transport across lymphatics, and ultimate delivery to lymph nodes. The key findings from this experimentation is as follows: i) the introduction of simulated transmural flow significantly and rapidly increases the uptake and transport efficiency of nanoparticles across lymphatic barriers, ii) rod shaped nanoparticles in the absence of interstitial flow were transported poorly across lymphatic barriers, but in the presence of flow had transport efficiency comparable to the spherical counterparts, iii) rod shaped nanoparticles were primarily transported paracellularly, iv) rod-shaped nanoparticles had improved lymph node delivery to lymph nodes in-vivo compared to similarly sized nanoparticles, and v) protein corona formation, particularly the presence of albumin, improved nanoparticle transport across LECs.

The nanoparticle shape has recently been shown to be a key characteristic that can improve drug delivery efficiency by improving transport across mucus and extracellular matrices, as well as improving circulation times. In the present study, 100 nm nanoparticles were stretched into 200 nm long rods with approximately 40-60 nm diameter narrow axis. When compared to similarly sized 200 nm spherical nanoparticles, the rod-shaped nanoparticles were transported more efficiently across lymphatic barriers and to lymph nodes. This was true especially in the presence of interstitial flow. This could be explained by previous findings indicating that rod shaped nanoparticles can align with flow. Indeed, one group was one of the first to observe that nanoparticle shape was a key factor in improving tumoral administration of nanoparticles. This improved delivery of nanoparticles within tissues has been hypothesized by them and other groups to be due to the fact rod-shaped nanoparticles can diffuse effectively through the extracellular matrix because the narrow axis and rod shape allows the particles to “roll” through the matrix if the matrix pore size is less than that of the nanoparticle. This improved transport through extracellular matrix is mirrored in the findings disclosed herein where rod shaped nanoparticles reached lymph nodes more effectively than similarly sized spherical nanoparticles. This could also explain the observed result where improved entry of the nanoparticle into the cortex of lymph nodes was observed after intradermal injection.

Groups have also examined how nanoparticle shape governs the uptake and transport mechanisms used to internalize nanoparticles. One group showed that micropinocytosis and macropinocytosis pathways were employed in the uptake of rod-shaped bio-nanoparticles in epithelial and endothelial cells. Another group also examined how nanoparticle shape effects uptake of rod-shaped nanoparticles in macrophages and the mechanisms driving uptake in macrophages. They found that clathrin and caveolin mediated endocytosis drove the uptake of 100 nm rod shaped nanoparticles. Other studies observed similar results where cellular mechanisms drove transport of rod-shaped nanoparticles into endothelial and epithelial cells. Collectively, these results on the cellular mechanisms governing rod shaped nanoparticle transport are replicated in the transport mechanism data, where inhibition of micropinocytotic and macropinocytotic transport with Dynasore and amiloride, respectively, hindered nanoparticle transport across lymphatics.

Protein corona formation after administration has been shown to be a key factor in nanoparticle drug delivery. In this study, insights were provided on how this corona formation impacts transport across lymphatics. There exists data indicating that the formation of the protein corona on the surface of nanoparticles is sometimes beneficial, and sometimes harmful for nanoparticle-based drug delivery. Some studies have demonstrated that the formation of protein corona on the surface of nanoparticles can hinder nanoparticle transport across endothelial and epithelial barriers by blocking nanoparticle interactions with the cell membrane. To combat this, many formulation strategies, including the one outlined herein employ PEG to impart stealth characteristics to nanoparticles. However, studies have shown that the addition of PEG can alter the formation of protein corona on the surface of nanoparticles. One study demonstrated that even at low PEG density apolipoprotein A1 and clusterin stealth proteins and that high PEG density is required to prevent protein adsorption. Another group created a library of different nanoparticles with combinations of PEG terminal groups: positively charged amines, negatively charged carboxyl groups, a combination of the two (zwitterionic), or non-charged methoxy groups. They found that amine-coated nanoparticles presented faster and higher protein absorption, and lower stability in serum. Carboxyl-PEG and methoxy-PEG Nanoparticles showed lower PC assembly. Interestingly, the lowest protein absorption was observed in zwitterionic-PEG Nanoparticles. One study also demonstrated that protein adsorption on PLGA- and PLGA-PEG-NP didn't depend on NP size within the range of 100 and 200 nm, however, PEGylation led to a significant reduction in protein corona formation.

These above results correlate with the findings disclosed herein where the charged nanoparticles, hydrophobic PS nanoparticles displayed the most significant protein corona formation compared to the PEGylated nanoparticles. An interesting consequence of this increased protein adsorption on the surface of PS nanoparticles is that when incubated in albumin, the presence of a pre-formed protein corona significantly improves nanoparticles transport efficiency. Of note, groups have generated numerous lymphatic vessel and lymph node targeting vaccine assemblies employing the innate albumin shuttling of lymphatics.

In summary, the data disclosed herein indicates that rod shaped nanoparticles can be used as a lymphatic drug delivery platform, and that albumin adsorption on the surface of nanoparticles could improve lymphatic drug delivery. This study is one of the first to examine how lymphatics regulate the transport of rod-shaped nanoparticles and on the delivery of rod-shaped nanoparticles to lymph nodes via lymphatic vessels.

What is claimed is:

1. A nanoparticle comprising an albumin coating and/or a rod-shape wherein said nanoparticle is targeted to the lymphatic system and/or lymph node in a subject.

2. The nanoparticle of claim 1, further comprising a PEG coating.

3. The nanoparticle of claim 1, further comprising a payload.

4. The nanoparticle of claim 3, wherein the payload is selected from the group consisting of a therapeutic agent; a prophylactic agent, diagnostic agent, anti-inflammatory agent; chemotherapeutic reagent; toxin, antigen and hapten.

5. The nanoparticle of claim 4, wherein the payload is an antigen or hapten for use in stimulation of an immune response.

6. The nanoparticle of claim 1, wherein the payload is releasable.

7. A medicament delivery system, or pharmaceutical composition, comprising the nanoparticle of claim 1 and a pharmaceutically acceptable carrier or excipient.

8. An immunogenic composition comprising the nanoparticle of claim 5.

9. The immunogenic composition of claim 8, wherein said composition is used as a vaccine.

10. The immunogenic composition of claim 8, further comprising an adjuvant.

11. The immunogenic composition of claim 8, wherein the antigen or hapten is derived from an allergen or self-antigen.

12. The immunogenic composition of claim 8, wherein the antigen or hapten is derived from a cancer cell.

13. The immunogenic composition of claim 8, wherein the antigen or hapten is derived from a pathogen.

14. Use of the nanoparticle of claim 1 for the manufacture of a medicament for treatment or preventing a disease or condition in a subject in need.

15. A vaccine composition comprising the immunogenic composition of claim 8.

16. The vaccine composition of claim 15, further comprising an adjuvant.

17. The use of claim 14, wherein the disease or condition is an immunological disease or condition.

18. A kit comprising a nanoparticle comprising a coating of albumin and/or a rod-shape.
